(19) United States
(12) Patent Application Publication
(10) Pub. No.: US 2020/0385775 A1
(43) Pub. Date: Dec. 10, 2020

(54) AUTOMATED YEAST BUDDING MEASUREMENT

(71) Applicant: Nexcelom Bioscience LLC, Lawrence, MA (US)

(72) Inventors: Leo L. Chan, North Andover, MA (US); Jean Qiu, Andover, MA (US); Peter Li, Andover, MA (US); Kevin Flanagan, Sterling, MA (US)

(21) Appl. No.: 17/000,285

(22) Filed: Aug. 22, 2020

Related U.S. Application Data
(62) Division of application No. 15/792,572, filed on Oct. 24, 2017.
Application No. 15/792,572 is a continuation of application No. 14/429,878, filed on Mar. 20, 2015, now abandoned.
(60) Provisional application No. 61/715,496, filed on Oct. 18, 2012.

(51) Int. Cl. C12Q 1/02; G01N 21/64; C12N 1/16; G01N 15/14

(52) U.S. Cl. CPC C12Q 1/02; G01N 21/6428; C12N 1/16; G01N 2015/0065; G01N 15/1475; G01N 2021/6441; G01N 2333/395; G01N 21/6458

(57) ABSTRACT

The invention generally relates to analyzing yeast viability and reproduction rate of yeasts. More particularly, the invention relates to efficient and effective methods and compositions for accessing and measuring budding percentages, viability and concentration of yeast cells.

### PRIORITY CLAIMS AND RELATED PATENT APPLICATIONS

This application is the U.S. national phase of and claims the benefit of priority from PCT/US13/64003, filed Oct. 9, 2013, which the benefit of priority from U.S. Provisional Application Ser. No. 61/715,496, filed on Oct. 18, 2012, the entire content of each of which is incorporated herein by reference in its entirety.

### TECHNICAL FIELDS OF THE INVENTION

The invention generally relates to analysis and measurement of yeast cells. More particularly, the invention relates to efficient and effective methods and compositions for assessing and measuring yeast budding, viability and concentration of yeast cells.

### BACKGROUND OF THE INVENTION

The biofuel and brewery industries have been utilizing baker's yeast (Saccharomyces cerevisiae) as the primary organism to commence fermentation process that produces CO₂ and bioethanol for their products. Currently, the largest biofuel process relies heavily on ethanol production, which utilizes Saccharomyces cerevisiae to perform fermentation on sugar cane, corn meal, polysaccharides, and waste water. Due to their high ethanol tolerance, final ethanol concentration, glucose conversion rate, and the historical robustness of industrial fermentation, yeasts are the ideal component for bioethanol production. (Antoni, et al. 2007 Appl. Microbiol. Biotech. vol. 77, pp. 23-35; Vertès, et al. 2008 J. Mol. Microbiol. and Biotech. vol. 15, pp. 16-30; Basso, et al. 2008 FEMS Yeast Res. vol. 8, pp. 1155-1163; Nikolić, et al. 2009 J. Chem. Technolog. Biotech. vol. 84, pp. 497-503; Gibbons, et al. 2009 In Vitro Cell. & Developm. Biol.—Plant, vol. 45, pp. 218-228; Hu, et al. 2007 Genetics, vol. 175, pp. 1479-1487; Argueso, et al. 2009 Genome Res. vol. 19, pp. 2258-2270; Eksteen, et al. 2003 Biotech. and Bioeng. vol. 84, pp. 639-646.)

Yeast budding is one of the most important parameters that breweries and biofuel companies use to determine the quality of the fermentation. Yeast pitching time for propagation and fermentation is the percentage of budding yeasts in the sample, which is known to estimate growth rate of yeast. Currently, there is no existing simple automated yeast budding detection method. Image flow cytometers have been used to perform yeast cell cycle to measure budding percentages. (Meredith, et al. 2008 Cytometry Part A, 73A: 825-833.) Image flow cytometers, however, are relatively expensive and require considerable amount of maintenance as well as highly trained technician for operation. Therefore, it is not suited for quality assurance in an industrial production setting. In addition, flow based sample preparation does not work with the biofuel samples due to the large corn mash debris in the sample that would clog the fluidics in the system.

Conventional analytical methods for concentration, viability and yeast budding percentages involve manual counting of yeasts particles in a hemacytometer under conventional light microscopy and colony counting of colony forming units in plating. These methods are tedious and time-consuming and are inherently inconsistent due to operator subjectivity. In order to obtain an accurate representation of the behavior of yeast during fermentation, an automated method for measuring concentration, viability, and budding percentage of the sample are required.

Therefore, there is an unmet need for an automated method for accurate and efficient measurement of yeast concentration, viability and budding percentage.

### SUMMARY OF THE INVENTION

The invention is based, in part, on the discovery of efficient and effective methods for automated measurement of yeast budding percentage. The present invention addresses the shortcomings of the previous methods in that real-time samples such as those from biofuel and wine production plants may be readily and accurately analyzed by the methods of the invention. Messy samples can be effectively measured as the invention allows high staining specificity. The automated, image-based cytometry method of the invention greatly simplifies the measurement process for the biofuel and brewery industries because it allows quick, accurate and concurrent determination of yeast budding, concentration and viability.

In one aspect, the invention generally relates to a method for automated analysis of budding status of yeast cells. The method includes: staining a sample to be analyzed for yeast cell budding with a dye in a buffer solution; acquiring a fluorescent image of the dye-stained sample; analyzing the fluorescent image of the dye-stained sample to determine the aspect ratio of the images of yeast cells in the dye-stained sample by a computer-based automated process, thereby determining the status of budding yeast cells in the sample.

In another aspect, the invention generally relates to a method for simultaneously determining yeast budding and viability. The method includes: staining a sample to be tested with a first dye and with a second dye in a buffer solution; acquiring a first fluorescent image of the sample stained with the first and second dyes, the first fluorescent image corresponding to the fluorescence from the first dye; acquiring a second fluorescent image of the sample stained with the first and second dyes, the second fluorescent image corresponding to the fluorescence from the second dye; analyzing the first fluorescent image to determine the aspect ratio of yeast cells by a computer-based automated process, thereby determining the status of budding yeast cells in the sample; and analyzing the second fluorescent image to determine yeast viability.

In yet another aspect, the invention generally relates to a method for simultaneously measuring concentration, viability, budding percentage of yeast cells in a sample. The method includes: staining a sample to be tested with a first dye and with a second dye under a buffer condition having a pH of about 5 to about 12; acquiring a first fluorescent image of the sample stained with the first and second dyes, the first fluorescent image corresponding to the fluorescence from the first dye; acquiring a second fluorescent image of the sample stained with the first and second dyes, the second fluorescent image corresponding to the fluorescence from the second dye; and analyzing the first and second fluorescent images to determine the concentration, viability, and budding percentage of yeast cells.

### BRIEF DESCRIPTION OF THE DRAWINGS

FIG. 1 depicts certain exemplary results from an embodiment of the method according to the invention, showing imaging analysis method for enumerating budding yeasts.

FIG. 2 depicts certain exemplary results from an embodiment of the method according to the invention, showing budding percentages measured from a growing yeast population.

FIG. 3 depicts certain exemplary results from an embodiment of the method according to the invention, showing comparisons with traditional manual counting method.

### DETAILED DESCRIPTION OF THE INVENTION

The present invention addresses the shortcomings of the previous methods and provides real-time and accurate analysis on a variety of samples such as those from biofuel plants that contain corn mash and other debris. Due to the high staining specificity, messy samples can be effectively measured. The invention also offers great efficiency and effectiveness by allowing simultaneous analysis and measurement of viability and concentration of yeast cells.

Recently, a novel imaging cytometry method has been developed by Nexcelom Bioscience (Lawrence, Mass.), which allows rapid measurement of cell concentration using inexpensive disposable counting chambers that require only 20 μl of samples. (Lai, et al. 20091 Clin. Oncology vol. 27, pp. 1235-1242; Nott, et al. 2009 J. Biol. Chem. vol. 284, pp. 15277-15288; Qiao, et al. 2009 Arteriosclerosis Thrombosis and Vascular Biol. vol. 29, pp. 1779-U139; Rounbehler, et al. 2009 Cancer Res. vol. 69, pp. 547-553; Shanks, et al. 2009 Appl. and Envir. Microbiol. vol. 75, pp. 5507-5513; Stengel, et al. 2009 Endocrinology, vol. 150, pp. 232-238.)

Utilizing combined bright-field and fluorescent imaging, the system allows automated cell image acquisition and processing using a novel counting algorithm for accurate and consistent measurement of cell population and viability on a variety of cell types. Applications such as enumeration of immunological, cancer, stem, insect, adipocytes, hepatocytes, platelets, algae, and heterogeneous cells, quantification of GFP transfection, viability using Trypan Blue or Propidium Iodide, measuring WBCs in whole blood, have been previously reported. More importantly, the method has been shown to produce consistent concentration and viability measurements of pure yeast for quality control purposes in biofuel, beverage, and baking industry. (Nexcelom Bioscience, “Simpe, Fast and Consistent Determination of Yeast Viability using Oxonol,” in Application Focus: Cellometer Vision 10X, pp. 1-2.)

Disclosed herein is a novel imaging fluorescence cytometry method employing the Cellometer® Vision (Nexcelom Bioscience, Lawrence, Mass.) for determining yeast budding, concentration and viability, for example, in corn mash from operating fermenters. Using a dilution buffer of the invention and staining the sample with Acridine Orange (AO) and Propidium Iodide (PI), the budding status, viable and nonviable yeasts are selectively labeled while nonspecific fluorescent signals from corn mash are eliminated. This method can efficiently perform yeast quality control using samples directly from processing fermenters without further filtration treatment, which can have a dramatic impact on monitoring consistent bioethanol production in the United States. Besides corn mash, viability of yeast in sugar cane fermentation can also be measured using this method. The method can also be readily applied to quality control in brewery production processes.

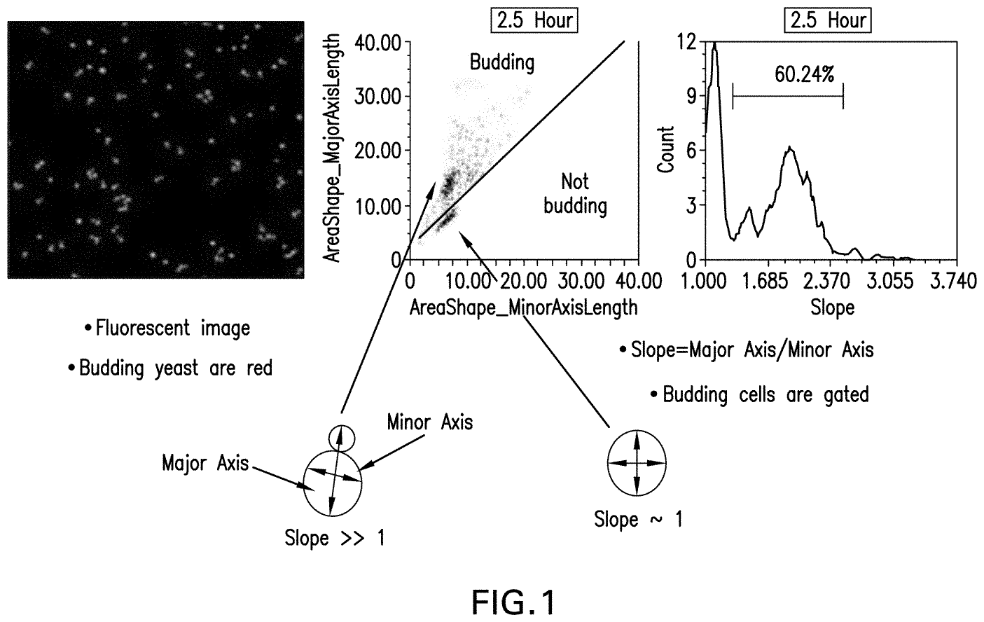

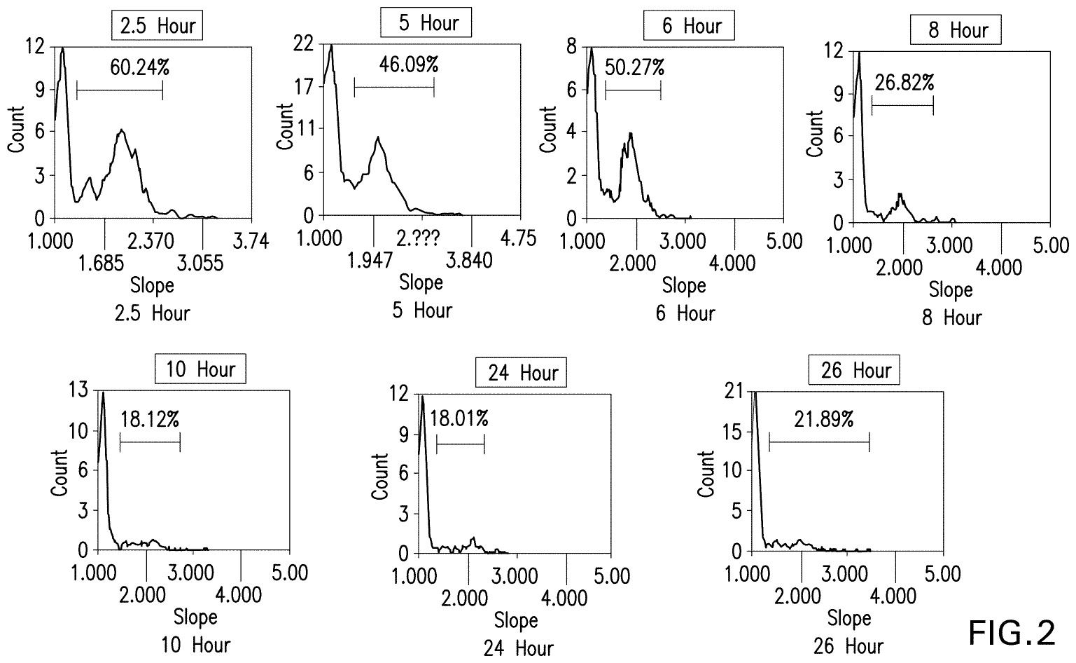

As depicted in FIG. 1, the invention utilizes a system that includes an automated microscopy, fluorescent stains and buffer, and an image analysis method. The image analysis aspect of the invention utilizes captured images of fluorescently stained-yeasts, and measures the major and minor axis length of each yeast particle. The ratio of major to minor axis length, defined herein as “slope=major/minor”, provides a variable (parameter) by which the budding status of yeast can be assessed. For instance, if the yeast particle is budding, then the major axis length will be greater than the minor axis length, thus producing a slope value greater than 1, whereas if the yeast particle is non-budding, the slope is to have a value of about 1 for a round shaped yeast. Using this parameter one can automatically gate the second population in FIG. 2 as the budding population.

In one aspect, the invention generally relates to a method for automated analysis of budding status of yeast cells. The method includes: staining a sample to be analyzed for yeast cell budding with a dye in a buffer solution; acquiring a fluorescent image of the dye-stained sample; analyzing the fluorescent image of the dye-stained sample to determine the aspect ratio of the images of yeast cells in the dye-stained sample by a computer-based automated process, thereby determining the status of budding yeast cells in the sample.

In certain preferred embodiments, the computer-based automated process includes automated measurement of the shape of budding yeasts in the sample. The threshold may be set such that a yeast cell (normally round shaped) is considered budding if its aspect ratio is 1.1 or greater. Other threshold may be set dependent on the application, for example at aspect ratio of 1.15 or greater, 1.2 or greater, 1.25 or greater, etc.

The dye may be any dye suitable for staining and analysis, for example, one or more selected from selected from the group consisting of Acridine Orange, SYTO 9, DAPI, Hoechst, Calcofluor White, Propidium Iodide, Ethidium Bromide, Oxonol, Mg-ANS, Acriflavine, ConA-FITC. The amount/concentrations of dyes used are dependent on the applications at hand. In the case of Acridine Orange, for example, a concentration may be in the range from about 1 μg/mL to about 50 μg/mL (e.g., about 2 μg/mL to about 50 μg/mL, about 5 μg/mL to about 50 μg/mL, about 10 μg/mL to about 50 μg/mL, about 20 μg/mL to about 50 μg/mL, about 25 μg/mL to about 50 μg/mL, about 1μg/mL to about 40 μg/mL, about 1μg/mL to about 30 μg/mL, about 1 μg/mL to about 20 μg/mL, about 1μg/mL to about 10 μg/mL).

The buffer may be any suitable buffer solution, for example, with a pH in the range from about 5 to about 12 (e.g., in a range from about 6 to about 12, from about 7 to about 12, from about 8 to about 12, at about 8, 9, 10, 11 or 12).

Any suitable samples may be analyzed by the method disclosed herein. For example, the sample may be one from a process of alcohol production using yeast. In certain embodiments, the sample to be tested is a sample from a biofuel fermentation process. The sample to be tested may contain certain debris, such as one or more of corn mash, sugar cane, cellulose and corn stover.

The methods of the invention is suitable for analyzing and measuring samples from the biofuel fermentation process producing one or more of ethanol, butanol and methanol from biomass.

Other examples of samples suitable for analysis by the disclosed methods include samples from a wine production process.

The methods are generally suitable for measuring budding status of yeast in general. Exemplary species of yeast include Saccharomyces cerevisiae.

In another aspect, the invention generally relates to a method for simultaneously determining yeast budding and viability. The method includes: staining a sample to be tested with a first dye and with a second dye in a buffer solution; acquiring a first fluorescent image of the sample stained with the first and second dyes, the first fluorescent image corresponding to the fluorescence from the first dye; acquiring a second fluorescent image of the sample stained with the first and second dyes, the second fluorescent image corresponding to the fluorescence from the second dye; analyzing the first fluorescent image to determine the aspect ratio of yeast cells by a computer-based automated process, thereby determining the status of budding yeast cells in the sample; and analyzing the second fluorescent image to determine yeast viability. The method may further include the step of analyzing the first and second fluorescent images to determine concentration of budding yeast cell.

In the case of Acridine Orange, for example, a concentration may be in the range from about 1 μg/mL to about 50 μg/mL (e.g., about 2 μg/mL to about 50 μg/mL, about 5 μg/mL to about 50 μg/mL, about 10 μg/mL to about 50 μg/mL, about 20 μg/mL to about 50 μg/mL, about 25 μg/mL to about 50 μg/mL, about 1 μg/mL to about 40 μg/mL, about 1 μg/mL to about 30 μg/mL, about 1 μg/mL to about 20 μg/mL, about 1 μg/mL to about 10 μg/mL). Also in the case of Propidium Iodide, for example, a concentration may be in the range from about 1 μg/mL to about 50 μg/mL (e.g., about 2 μg/mL to about 50 μg/mL, about 5 μg/mL to about 50 μg/mL, about 10 μg/mL to about 50 μg/mL, about 20 μg/mL to about 50 μg/mL, about 25 μg/mL to about 50 μg/mL, about 1 μg/mL to about 40 μg/mL, about 1 μg/mL to about 30 μg/mL, about 1 μg/mL to about 20 μg/mL, about 1 μg/mL to about 10 μg/mL).

In certain embodiments, the first dye is selected from the group consisting of Acridine Orange, SYTO 9, DAPI, Hoechst, Calcofluor White and the second dye is selected from the group consisting of Propidium Iodide, Ethidium Bromide, Oxonol, Mg-ANS. In certain preferred embodiments, the first dye is Acridine Orange and the second dye is Propidium Iodide.

In yet another aspect, the invention generally relates to a method for simultaneously measuring concentration, viability, budding percentage of yeast cells in a sample. The method includes: staining a sample to be tested with a first dye and with a second dye under a buffer condition having a pH of about 5 to about 12; acquiring a first fluorescent image of the sample stained with the first and second dyes, the first fluorescent image corresponding to the fluorescence from the first dye; acquiring a second fluorescent image of the sample stained with the first and second dyes, the second fluorescent image corresponding to the fluorescence from the second dye; and analyzing the first and second fluorescent images to determine the concentration, viability, and budding percentage of yeast cells.

The developed automated yeast budding detection method can be applied to numerous type of yeasts. The measured slope parameter can be adjusted so that the restriction on the size of the bud can be fixed to remove the subjectivity between different technicians. This disclosed method is rapid and simple and can be easily adapted to a quality assurance setting at production or research facilities for the brewery and biofuel industries. Further adding to the uniqueness of the disclosed invention is that all three important parameters (yeast concentration, viability and budding percentages) can be measured simultaneously.

### EXAMPLES

### Yeast Preparation

A yeast growth culture was prepared by incubating yeast in YPD medium overnight at 30° C. The yeast culture (800 μL) was then re-suspended in a 20 mL medium glass tube by shaking at 30 ° C. The yeasts were collected at time points: 2.5, 5, 6, 8, 10, 24, and 30 hours and were stained with Acridine Orange and Propidium Iodide. The fluorescent images were captured.

### Automated Detection

At each time point, the fluorescent images were analyzed using Cell Profiler (Cambridge, Mass.) and Nexcelom Cellometer Software (Lawrence, Mass.), where the exported data was imported into FCS Express 4 Image (Los Angeles, Calif.). The FCS Express 4 was then used to plot the slope of each yeast particle so that the two populations (budding and non budding) are separated and measured (FIG. 1). This method was incorporated into the Cellometer® software so that the slope value was used to determine budding percentages, while the concentration and viability were measured simultaneously.

### Manual Comparison

At each time point, manual counting of yeast particles and budding are performed under bright-field imaging and fluorescent imaging. Total yeast particles and yeasts that are budding are manual counted to generate the budding percentage in the sample. The criterion was currently set that if two yeasts were touching, then it would be counted as one bud. The results of the manual counting were compared to the automated detection method.

### Validation of Automated Method

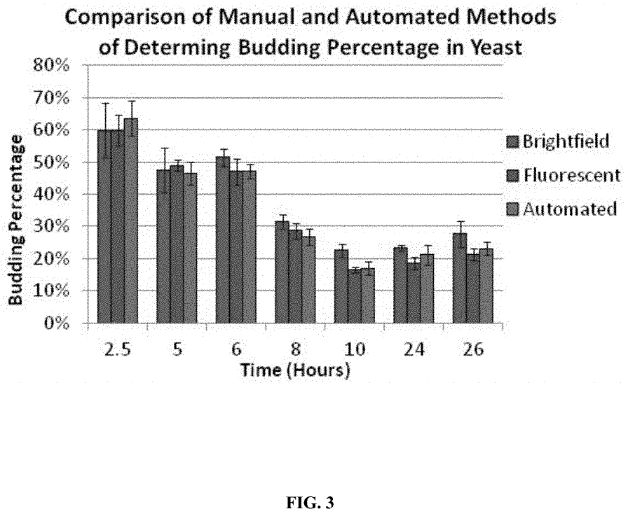

The gating results for the automated budding detection method at each time point are shown in FIG. 3. There was a clear trend where, in the beginning of the growth phase, high percentages of budding were observed. Then from the lag phase, log phase to station phase, the budding percentages decreased. The budding percentages decreased from ˜60 to 20% during the growth period.

The automated budding results were compared to the bright-field and fluorescent manual counting (FIG. 3). The results showed comparable budding percentages measured between all 3 methods. The bright-field manual counting typically over estimate the budding due to counting as debris, whereas the fluorescent manual counting method stayed relatively consistent with the automated method.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The examples herein contain important additional information, exemplification and guidance that can be adapted to the practice of this invention in its various embodiments and equivalents thereof.

1-46. (canceled)

47. A method for simultaneously determining yeast budding and viability, comprising:
staining a sample to be tested with a first dye and with a second dye in a buffer solution;
acquiring a first fluorescent image of the sample stained with the first and second dyes, the first fluorescent image corresponding to the fluorescence from the first dye;
acquiring a second fluorescent image of the sample stained with the first and second dyes, the second fluorescent image corresponding to the fluorescence from the second dye;
analyzing the first fluorescent image to determine the aspect ratio of yeast cells by a computer-based automated process, thereby determining the status of budding yeast cells in the sample; and
analyzing the second fluorescent image to determine yeast viability.

48. The method of claim 47, wherein the computer-based automated process comprises image analysis to measure the shape of budding yeasts.

49. The method of claim 47, wherein an image of yeast cell is considered budding if its aspect ratio is 1.1 or greater.

50. The method of claim 47, wherein the first dye is selected from the group consisting of Acridine Orange, SYTO 9, DAPI, Hoechst, Calcofluor White and the second dye is selected from the group consisting of Propidium Iodide, Ethidium Bromide, Oxonol, Mg-ANS.

51. The method of claim 50, wherein the first dye is Acridine Orange and the second dye is Propidium Iodide.

52. The method of claim 47, wherein the buffer condition has a pH of about 5 to about 12

53. The method of claim 47, wherein the sample to be tested is a sample from a biofuel fermentation process, a wine production process or a beer brewing production process.

54. The method of claim 63, wherein the biofuel fermentation process comprises producing ethanol, butanol or methanol.

55. The method of claim 63, wherein the sample to be tested comprises debris of corn mash, sugar cane, cellulose or corn stover.

56. The method of claim 47, wherein the yeast is the species of Saccharomyces cerevisiae.

57. The method of claim 51, wherein Acridine Orange is at a concentration of about 1 μg/mL to about 50 μg/mL and Propidium Iodide is at a concentration of about 1 μg/mL to about 50 μg/mL.

58. The method of claim 47, further comprising analyzing the first and second fluorescent images to determine concentration of budding yeast cell.

59. A method for simultaneously measuring concentration, viability, budding percentage of yeast cells in a sample, comprising:
staining a sample to be tested with a first dye and with a second dye under a buffer condition having a pH of about 5 to about 12;
acquiring a first fluorescent image of the sample stained with the first and second dyes, the first fluorescent image corresponding to the fluorescence from the first dye;
acquiring a second fluorescent image of the sample stained with the first and second dyes, the second fluorescent image corresponding to the fluorescence from the second dye; and
analyzing the first and second fluorescent images to determine the concentration, viability, and budding percentage of yeast cells.
